(19) United States
(12) Patent Application Publication
(10) Pub. No.: US 2018/0050213 A1
(43) Pub. Date: Feb. 22, 2018

(54) HOUSING FOR MEDICAL IMPLANT

(71) Applicant: Forschungszentrum Juelich GmbH, Juelich (DE)

(72) Inventor: Christian Hauptmann, Starnberg (DE)

(21) Appl. No.: 15/319,558

(22) PCT Filed: May 28, 2015
(86) PCT No.: PCT/EP2015/061908

§ 371 Date: Dec. 16, 2016

(30) Foreign Application Priority Data
Jun. 18, 2014 (DE) 10 2014 009 136.8

(51) Int. Cl. A61N 1/375

(52) U.S. Cl. CPC A61N 1/3756; A61N 1/3752

(57) ABSTRACT

The invention relates to a housing for a medical implant. According to the invention, at least part of a housing for a medical implant is made of an insulator material as a dielectric, and the inner face and outer face of the insulator material each include at least one electrically conducting contact area (2, 2a, 2b . . . 3, 3a, 3b . . . ) which acts as a capacitor electrode and forms a capacitor along with the insulator material.

### CROSS REFERENCE TO RELATED APPLICATIONS

This application is a National Stage of PCT No. PCT/EP2015/061908, filed on May 28, 2015, which claims the benefit of German Application No. 10 2014 009 136.8, filed on Jun. 18, 2014, the contents of each of which are hereby incorporated by reference.

### TECHNICAL FIELD

The invention relates to a housing for a medical implant.

### BACKGROUND

Diseases of the central nervous system such as epilepsy, Parkinson's disease or obsessive compulsive disorders are inter alia treated by means of direct electrical stimulation of the brain. For this purpose, electrodes are implanted into the target areas and are electrically connected to corresponding implant systems beneath the skin. Electrical stimuli are transmitted to the target area via these implant systems. In electrical stimulation, the observation of the charge density, and thus of the charge quantity per pulse, is in particular an important criterion in order to avoid permanent damage to the tissue in the course of the therapeutic stimulation. Devices for deep brain stimulation are described in “Deep Brain Stimulation Devices: A Brief Technical History and Review”, Robert J. Coffey, 2008, Artificial Organs 33(3), 208-220.

In a similar manner, cardiac arrhythmia is, for example, treated by pacemakers with which implants are likewise inserted into the body.

In accordance with the prior art, the transfer of the charge quantity is limited by a coupling capacitor. A coupling capacitor is, for example, required per stimulation contact. The capacity typically amounts to 100 nF and more, for example 1000 nF, in order to limit the charge transfer to 1 μC, for example.

Newer electrodes in accordance with the prior art provide a large number of electrode contacts, for example 40 or 80 contacts, such as are, for example, described by H. C. F. Martens et. al in “Spatial steering of deep brain stimulation volumes using a novel lead design”, Clinical Neurophysiology, (2011), 122, 558-566.

To date, individual capacitors or an array of capacitors are typically used for implementing the coupling capacitors. The capacitors are usually ceramic-based capacitors having a capacity of 100 nF or more, for example. The size of the capacity is substantially determined by the supply voltage of the implant, by the surface of the stimulation contacts and by the demands on the efficiency of the implant. If a higher supply voltage or a smaller contact surface is selected, the capacity can, for example, be selected smaller. The cable feedthrough from the interior of the implant to the connectors of the electrode is frequently implemented by integrating a ceramic component into an opening of the housing which is usually composed of titanium. Ceramic disks are pressed in the titanium housing in conventional cable feedthroughs.

The apparatus in accordance with the prior art have the disadvantage that a large number of components have to be used, wherein high space requirements exist. The location of the cable feedthrough furthermore represents a critical region, which can be the location of a leak and thus represents a safety risk, at which complications or even damage to the patient can occur due to the penetration of body fluid.

With the apparatus in accordance with the prior art, safety is impaired and the implants are large, which has the consequence of impairments in the use of the implants both for the patient and for the doctor. Problems in the construction arise for the engineer which can only be solved with difficulty. The construction size of the implant is substantially determined by the battery, the coupling capacitors, the electronics, the adapter and the cable feedthrough.

A large implant is visible from the outside due to its size. The large number of coupling capacitors take up a very large space within the implant and thus prevent the reduction in size of the implant in order, for example, to select a more favorable implantation site in the region of the skullcap. The large number of electrical contacts have to be led out of the inner space of the hermetically closed implant housing, which can result in greater difficulties with a large number of contacts and which hugely restricts the size and the construction shape of the implant. The larger the implant is, the higher the risk of an inflammatory reaction or of a rejection of the implant in the patient is.

### SUMMARY OF THE INVENTION

It is therefore an object of the invention to provide an implant which is reduced in size with respect to the implants available in accordance with the prior art and which allows a higher safety for the patient. The construction of implants should be facilitated for the engineers. In the patient, the implant should result in reduced health damage, such as inflammatory reactions, or in a reduced risk of rejection.

Starting from the preamble of claim 1, the object is satisfied in accordance with the invention by the features set forth in the characterizing portion of claim 1.

It is now possible with the housing in accordance with the invention to reduce the implant in size with respect to the prior art and to increase the operational safety for the patient. The risk for an entry of body fluids into the implant can in particular be reduced. A health risk for the patient, for example by inflammatory reactions or by the rejection of the implant, can be reduced. The construction is facilitated for the engineers who develop the implants.

Advantageous further developments of the invention are set forth in the dependent claims.

The invention will be described in its general form in the following.

### BRIEF DESCRIPTION OF THE DRAWINGS

The Figures show exemplary embodiments of the housing in accordance with the invention. There are shown:

FIG. 1: an embodiment with a planar arrangement of electrically conductive contact surfaces;

FIG. 2: an embodiment in which the electrically conductive contact surfaces extend into the housing in the shape of lamellae; and

FIG. 3: an embodiment in which the electrically conductive contact surfaces extend into the housing in a layer-like manner.

### DETAILED DESCRIPTION

In accordance with the invention, a housing is provided for an implant and is at least composed of an insulator as a dielectric in a part region, and preferably completely, said dielectric being a component of a capacitor, wherein at least one electrically conductive contact surface which forms a capacitor with the housing composed of insulator material is provided at the inner side and the outer side of the housing respectively. The contact points at the inner side and at the outer side of the housing should be disposed opposite one another as accurately as possible.

The housing in accordance with the invention has the functions of forming an electrical capacitor and of preventing an entering of body fluid into the implant.

The housing is preferably a housing for medically implantable pulse generators.

The inner and outer electrically conductive contact surfaces serve as a capacitor electrode and can be brought into contact with an electrical line which exerts a charge on them at the inner side of the housing and which results in a change of the charge at the outer side, said charge being able to be led off into a stimulation electrode by which an electrical stimulus should be exerted on the medical target area, for example on a brain region or the heart muscle. In an embodiment of the invention, no further stimulation electrodes have to be attached to the electrically conductive contact surfaces which are located at the outer side of the housing. This is, for example, the case when the implant can be implanted such that the contact surfaces themselves serve as stimulation electrodes.

If the outer electrically conductive contact surfaces do not themselves serve as stimulation electrodes, they have to be provided with a suitable insulation layer, for example composed of silicone.

The components which are necessary for the function of the implant, such as a battery and a control, are located in the housing with a line to the electrically conductive contact surface at the inner side of the implant.

In accordance with the invention, a housing is provided for a medical implant and has a preferred capacity of between 1 nF and 1000 nF in the construction section which is configured as a capacitor. The capacity in particular lies between 5 nF and 1000 nF, particularly preferably between 5 nF and 500 nF. The values result from the limits which are determined in that a charge quantity which should not exceed a value which damages the tissue is introduced into the biological tissue. The lower limit of the value of the capacity is determined by the charge quantity which is sufficient to achieve a physiological effect, namely, for example, to sufficiently stimulate the heart muscle when the implant serves as a pacemaker or to apply electrical stimulus patterns in the brain which have a physiological effect, for example, the suppression of a neural activity or the reduction of a pathological activity.

All capacitors which result in values for capacities which satisfy this purpose should therefore by covered by the protected zone.

To avoid tissue damage, the charge density should not exceed a specific limit value. In accordance with the prior art, this limit value is, for example, specified at a maximum of 30 μC/cm². Together with the information on the dimension of the stimulation electrode surface for typical examples in accordance with the prior art, maximum charges of 1.8 μC can be derived from this for a stimulation electrode surface of 0.06 cm² and of 0.12 μC for a stimulation electrode surface of 0.004 cm². With an assumed maximum voltage of 10 Volt, 180 nF or 12 nF are thus calculated for the capacity. A good value of the capacity for small stimulation electrode surfaces is 10 nF.

A small stimulation electrode surface only allows the application of a small charge per pulse, whereas a larger charge per pulse can be applied with a larger stimulation electrode surface without risking tissue damage. In practice, larger capacities are used in implants in accordance with the prior art than justified by the safety assessment since larger capacities have a higher energy efficiency in the applications. These larger capacities can, however, also be implemented in the apparatus in accordance with the invention.

The construction design of the medical implant in accordance with the invention is based on the physical principles with which the capacities can be reached which should be implemented in accordance with the invention.

In accordance with formula (1),

C=ε₀εᵣA/d (1)

applies
where

C=capacity (F)

A=surface of the contact surface (m²)

ε₀=permittivity in a vacuum

εᵣ=relative permittivity of the insulator

d=distance of the inner contact surface from the outer contact surface which form the capacitor with the insulator (m)

Combinations of the size and distance of the electrically conductive contact surfaces and of the necessary permittivity of the insulator material can be determined by a suitable selection of the corresponding sizes for the parameters specified in formula (1), with the housing in accordance with the invention being able to be implemented by said combinations.

The skilled person is able to find dimensions and materials which are sensible for the construction of the housing on the basis of the relationship in accordance with formula (1).

The size of the electrically conductive contact surfaces can be in the order of magnitude of a few mm². A typical value is 9 mm², for example. The distance of the electrically conductive contact surfaces may not negatively impair the stability of the housing and can, for example, amount to 100 μm.

Material which has a permittivity of between 5 and 20,000 is preferably selected for the insulator from which the housing is produced.

The material should be biocompatible; that is, it should in particular be compatible for human tissue and not be toxic.

Ceramic materials can be used for this purpose.

By way of example but not in a limiting manner, the housings can comprise aluminum oxide ceramic material or a ceramic material of Yttria-stabilized zirconium oxide (Zr₂O₃Y₂O₃).

The ceramic materials which are already known in accordance with the prior art and which are inserted in the implant region can be used.

Suitable monocrystalline crystals can also be considered.

Materials can also be used which will be developed in the future and which have suitable properties.

Metallic elements or electrically conductive substances can be used as electrically conductive contact points. Platinum-iridium electrodes can thus be used, for example.

Any material can be used which is known as an electrode material in medicine and which comes into contact with the human body. Materials can also be used which will be developed in the future and which have suitable properties.

The electrically conductive contact points or contact surfaces can have different designs.

In the simplest embodiment of the housing in accordance with the invention for a medical implant, an electrically conductive contact point is located at the inner side of the housing and a further electrically conductive contact point is located at the outer side of the housing, said contact points preferably being planar and being arranged with respect to one another such that they form a capacitor having the above-mentioned capacity in conjunction with the insulator material of the housing.

A strictly planar design of the electrically conductive contact surfaces is not absolutely necessary. It is also possible for the electrically conductive contact surfaces to be arched.

The two electrically conductive contact surfaces can then each be provided with a line which can conduct the electrical signals.

Depending on the application, a plurality of electrically conductive contact surfaces can also be attached to the inner side and to the outer side which form a capacitor with the material of the housing. In each case, at least two respective electrically conductive contact points can thus be attached to the inner side and to the oppositely disposed outer side of the housing in accordance with the invention. The electrically conductive contact surfaces should be disposed opposite one another. The more they are displaced toward one another, the greater the deviation is in the oppositely disposed arrangement and the smaller the capacity becomes. The number of electrically conductive contact points which are located at the inner side and at the outer side is freely selectable and is determined by the application which should be carried out with the implant. For example, 1, 2, 3, 4, 8, 40 or 80 electrically conductive contact points which are each connected to an electrical line can be located at the inner side and at the outer side of the housing respectively.

The distance of the electrically conductive contact surfaces between one another at one side should be dimensioned such that no charge jumps from one electrically conductive contact surface to another electrically conductive contact surface.

In an example, four electrically conductive contact surfaces attached in the housing can thus be supplied with power by four lines and four further contact surfaces are located at the side of the housing disposed opposite the electrically conductive contact surfaces, with four lines which conduct the power to the target point of a stimulation electrode in turn being attached to said four further contact surfaces.

The electrically conductive contact surfaces should be dimensioned such that their dimensions are in the μm or mm range in dependence on how many contact points should be present.

The electrically conductive contact points can be attached to the surface of the housing, but they can in this respect also extend into the housing.

In an advantageous embodiment of the invention, the electrically conductive contact surfaces are optimized. For this purpose, the surfaces which become active as capacitor surfaces are enlarged. The electrically conductive contact surfaces can be arranged in different geometries with respect to one another for this purpose. Such arrangements and the methods by which they can be determined are known to the skilled person. The electrically conductive contact surfaces can, for example, be arranged in the shape of lamellae or in a layer-like manner. In the layer-like arrangement, parts of the electrically conductive contact points can extend into the housing material like spigots.

The housing can be configured such that it has the same thickness everywhere. The point of the housing at which the electrically conductive contact points are located can alternatively be thicker or thinner than at other points of the housing in accordance with the function as a capacitor.

The use of the housing in accordance with the invention for a medical implant, in particular for pulse generators, has the advantage that it is particularly stable mechanically, saves space and is particularly safe since there are no points at which fluid can enter the housing. The implants can be so small that they can even be installed into the skull. Exemplary dimensions are 8 mm×40 mm×30 mm. Health-damaging effects such as inflammatory reactions or a rejection of implants can be reduced or prevented.

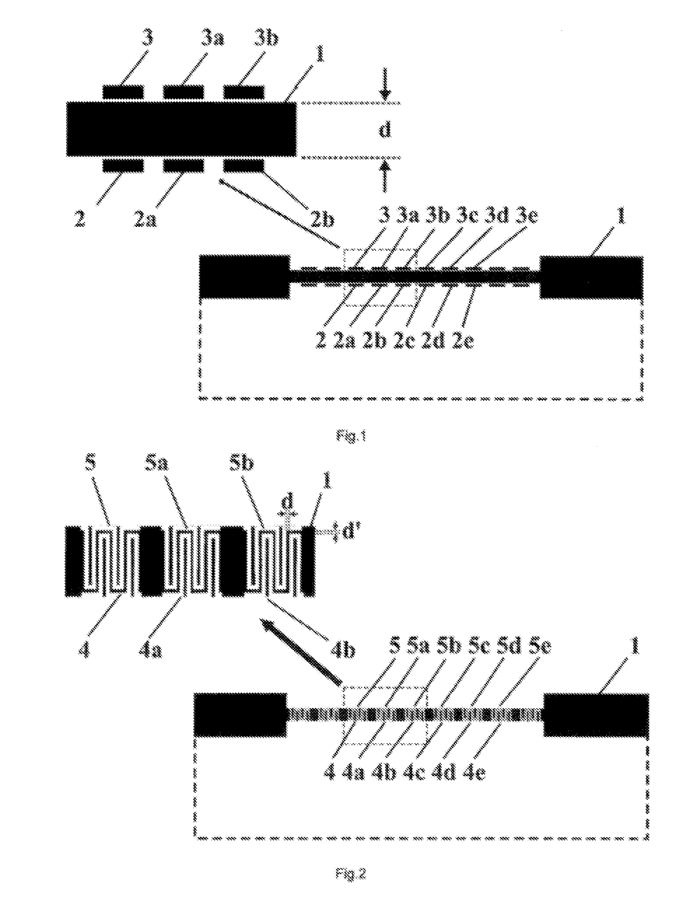

FIG. 1 shows a housing 1 for a medical implant which, in a region, has electrically conductive contact surfaces 2, 2a, 2b, 2c, 2d, 2e . . . at the inner side and electrically conductive contact surfaces 3, 3a, 3b, 3c, 3d, 3e . . . at the outer side, said electrically conductive contact surfaces being disposed opposite one another. A housing 1 is located therebetween and is configured as a dielectric which establishes a distance d between the contact surfaces. Electrical lines can be attached to each of the electrically conductive contact surfaces 2, 2a, 2b, 2c, 2d, 2e . . . and 3, 3a, 3b, 3c, 3d, 3e . . . , but are not shown in the Figure. The lines which are attached outside supply end points of stimulation electrodes with power. The lines which are attached inside are supplied with power by a control.

The housing is designated by 1 in FIG. 2. The electrically conductive contact surfaces 4, 4a, 4b, 4c, 4d, 4e . . . at the outer side and 5, 5a, 5b, 5c, 5d, 5e . . . at the inner side of the housing 1 are formed in the shape of lamellae and extend into the housing 1. The electrically conductive contact surfaces in this respect form serpentine lines which are shown in white, which extend at the surface of the housing 1 and which extend into the housing 1. The electrically conductive contact surfaces of the inner side and of the outer side of the housing 1 in this respect engage into one another like toothed wheels. This can be achieved in that the electrically conductive contact surfaces are introduced into the housing 1 during its production. FIG. 2 represents a cross-section such that the lamella-like representation is a projection onto the edge of planar, curved electrically conductive contact surfaces which are partly arranged in parallel with one another in the interior of the housing material. The distance d, d′ between the electrically conductive contact points formed in the shape of plates is shown in the enlarged section of FIG. 2. The distances d and d′ can be of the same size, but can also be different.

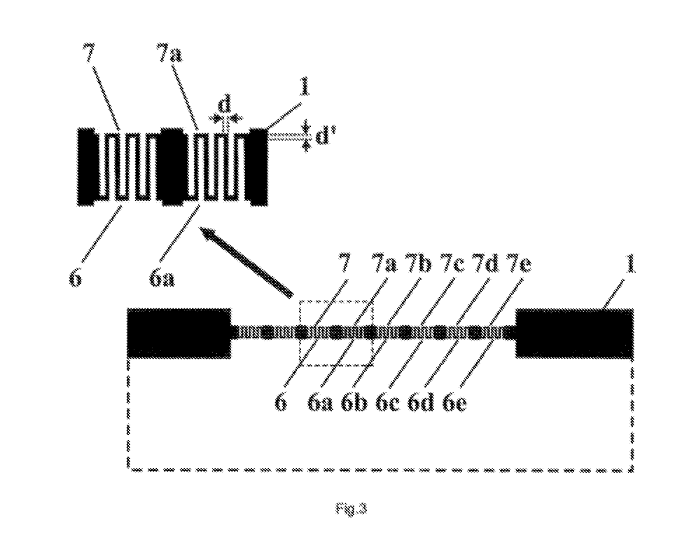

In FIG. 3, the electrically conductive contact points 6, 6a, 6b, 6c, 6d, 6e . . . and 7, 7a, 7b, 7c, 7d, 7e . . . are configured as plates which are located at the inner side and at the outer side of the housing 1 and which extend into the housing 1 in the form of surfaces which result in a parallel, layer-like arrangement of the electrically conductive contact surfaces. The electrically conductive contact points are in turn represented as a projection onto the section of the housing 1 such that the visible white lines represent the projection onto the side of band-shaped electrically conductive contact points. The electrically conductive contact points alternately extend into the housing 1 and form plates which are arranged in parallel with one another and whose distance d, d′ determines the capacity of the capacitor. The distances d and d′ can be the same or different.

### EXAMPLE

Calculation of the necessary size of the surface, of the metallic contact surfaces and of the thickness of the ceramic material.

Basic assumptions: the relative permittivity εᵣ of the ceramic material corresponds to that of barium titanate (BaTiO₃): up to 10,000. Assumption 1: an electrically conductive contact surface of 9 mm² (planar) or 30 mm² (layer-wise) is available per contact. A thickness of the ceramic layer of approx. 80 μm (or 270 μm) results from this in accordance with the formula C=ε₀ εᵣ A/d for achieving a capacity C=100 nF. Smaller capacities can also be sufficient for smaller electrode contact surfaces; the minimum thickness of the ceramic layer can thereby increase, which facilitates the technical implementation and increases the mechanical stability.

1-5. (canceled)

6. A medical implant, comprising:
a housing including:
an insulator material as a dielectric in a part region and the insulator material respectively having at least one electrically conductive contact surface at an inner side and an outer side of the housing, with the at least one electrically conductive contact surface acting as a capacitor electrode and forming a capacitor together with the insulator material,
wherein there is no electrically conductive connection between the at least one electrically conductive contact surface at the inner side of the housing and the at least one electrically conductive contact surface at the outer side of the housing,
wherein the at least one electrically conductive contact surface at the inner side of the housing has lamellae or plates which extend into the insulator material from the inner side of the housing,
wherein the at least one electrically conductive contact surface at the outer side of the housing has lamellae or plates which extend into the insulator material from the outer side of the housing, and
wherein the lamellae or plates of the at least one electrically conductive contact surface at the inner side of the housing and the lamellae or plates of the at least one electrically conductive contact surface at the outer side of the housing are arranged alternately and form the capacitor;
a control that is accommodated within the housing and that is connected to the at least one electrically conductive contact surface at the inner side of the housing in order to supply power to said housing; and
at least one stimulation electrode that is located outside the housing and that is electrically conductively connected to the at least one electrically conductive contact surface at the outer side of the housing.

7. The medical implant in accordance with claim 6, wherein the capacitor has a capacity of between 5 nF and 1000 nF.

8. The medical implant in accordance with claim 6, wherein the insulator material is a ceramic material.

9. The medical implant in accordance with claim 6, wherein the electrically conductive contact surfaces comprise a platinum-iridium alloy.

10. The medical implant in accordance with claim 6, wherein the housing respectively has at least 2 to 80 contact surfaces at the inner side and at the outer side.
